(19) United States
(12) Patent Application Publication
(10) Pub. No.: US 2012/0282359 A1
(43) Pub. Date: Nov. 8, 2012

(54) Compositions, Kits, and Methods for the Treatment of Conditions Associated with Elevated Cholesterol Levels

(76) Inventors: Abel Ennio Moreyra, Piscataway, NJ (US); Ashraf M. Koraym, East Brunswick, NJ (US); Alan Chaney Wilson, North Brunswick, NJ (US); Owen Rickford Carryl, Mason, OH (US)

(21) Appl. No.: 13/455,595

(22) Filed: Apr. 25, 2012

Related U.S. Application Data
(63) Continuation of application No. 10/979,329, filed on Nov. 2, 2004, now abandoned.
(60) Provisional application No. 60/518,183, filed on Nov. 7, 2003.

(51) Int. Cl. A61K 36/68; A61K 31/215; A61P 3/00; A61K 31/702; A61K 36/899; A61K 31/366; A61K 31/40

(52) U.S. Cl. 424/738; 514/460; 514/510; 514/423; 514/54; 424/750

(57) ABSTRACT

Disclosed herein are methods of treating a condition associated with elevated cholesterol levels are provided, comprising administering to a mammal in need of such treatment a safe and effective amount of a cholesterol biosynthesis inhibitor and a soluble fiber. Further disclosed herein are kits comprising a first composition comprising a cholesterol biosynthesis inhibitor selected from the group consisting of HMG CoA reductase inhibitors, HMG CoA synthase inhibitors, and mixtures thereof; and a second composition comprising a soluble fiber. Even further described are compositions comprising a cholesterol biosynthesis inhibitor selected from the group consisting of HMG CoA reductase inhibitors, HMG CoA synthase inhibitors, and mixtures thereof; and a soluble fiber.

### CROSS-REFERENCE TO RELATED APPLICATION

This application is a continuation of U.S. patent application Ser. No. 10/979,329, filed on Nov. 2, 2004, which claims the benefit of U.S. Provisional Application No. 60/518,183, filed on Nov. 7, 2003.

### FIELD OF THE INVENTION

The present invention relates to compositions, kits, and methods that are useful for treatment of conditions associated with elevated cholesterol levels.

### BACKGROUND OF THE INVENTION

The class of compounds known as cholesterol biosynthesis inhibitors, including HMG CoA reductases inhibitors (including a class commonly referenced as “statins”) are commercially utilized to effect the reduction of cholesterol levels in the mammalian system. Commercially available products for human use include MEVACOR (comprising lovastatin, Merck), PRAVACHOL (comprising pravastatin, Bristol Myers Squibb), LESCOL (comprising fluvastatin, Novartis), ZOCOR (comprising simvastatin, Merck), and LIPITOR (comprising atorvastatin, Pfizer). These products have proven useful, resulting in lowering LDL-cholesterol levels by about 25% to about 45%, although certain products are reported to provide higher reductions about the level of 60% when administered at high dose. However, with a few exceptions, these products have relatively low peroral bioavailability. Even further, some patients cannot tolerate statins at high dose.

Moreover, these products have little if any effect upon the absorption of dietary cholesterol, absorption of endogenous biliary cholesterol, and resorption of bile acids. Bile acid is synthesized by the liver using cholesterol. As such, it would be beneficial to provide a method of lowering LDL-cholesterol levels while currently absorbing dietary cholesterol, absorbing biliary cholesterol and/or binding bile acid (which, in turn, could result in the uptake of further cholesterol from the circulation to synthesize further bile acid). Along with increased lowering of LDL cholesterol and other components, decreased dose or frequency of statins could be realized.

There are a vast variety of approaches that could be investigated in an effort to reach such an objective, however, limited success has heretofore been reported. For example, a combination therapy of ezetimibe and simvastatin is the current subject of a regulatory approval process in the United States. Other approaches are not known to have reached any prominence to date. Certain soluble fibers have been reported to provide a benefit in reduction of cholesterol levels. For example, psyllium and oat fiber have been particularly referenced as having beneficial effects. However, to date, there have been no reports or even suggestions that such fibers have efficacies that exceed, or even parallel, that of statins. To this end, combination therapies have not been suggested in the literature as it would have been expected that such therapies would produce negligible benefits.

However, it is the surprising and exciting discovery of the present inventors that a combination therapy of a cholesterol biosynthesis inhibitor and a soluble fiber provide truly synergistic results relative to either of these components alone. These results are indeed unexpected and now present opportunities to revolutionize cholesterol-lowering therapies in humans and other mammals.

### SUMMARY OF THE INVENTION

In one embodiment of the present invention, methods of treating a condition associated with elevated cholesterol levels are provided, comprising administering to a mammal in need of such treatment a safe and effective amount of a cholesterol biosynthesis inhibitor and a soluble fiber.

In another embodiment of the present invention, kits are provided comprising:

(a) a first composition comprising a cholesterol biosynthesis inhibitor selected from the group consisting of HMG CoA reductase inhibitors, HMG CoA synthase inhibitors, and mixtures thereof; and

(b) a second composition comprising a soluble fiber.

In yet another embodiment of the present invention, compositions are provided comprising:

(a) a cholesterol biosynthesis inhibitor selected from the group consisting of HMG CoA reductase inhibitors, HMG CoA synthase inhibitors, and mixtures thereof; and

(b) a soluble fiber.

### DETAILED DESCRIPTION OF THE INVENTION

Various documents including, for example, publications and patents, are recited throughout this disclosure. All such documents are hereby incorporated by reference.

All percentages and ratios are calculated by weight unless otherwise indicated. All percentages and ratios are calculated based on the total composition unless otherwise indicated.

Referenced herein are trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

In the description of the invention various embodiments and/or individual features are disclosed. As will be apparent to the ordinarily skilled practitioner, all combinations of such embodiments and features are possible and can result in preferred executions of the present invention.

The compositions herein may comprise, consist essentially of, or consist of any of the elements as described herein.

While various embodiments and individual features of the present invention have been illustrated and described, various other changes and modifications can be made without departing from the spirit and scope of the invention. As will be also be apparent, all combinations of the embodiments and features taught in the foregoing disclosure are possible and can result in preferred executions of the invention.

Compositions and Components Utilized in the Present Invention The present invention relates to compositions, kits, and methods which utilize the combination of a cholesterol biosynthesis inhibitor and a soluble fiber. The compositions, kits, and methods are useful for the inhibition of cholesterol biosynthesis, coupled with the inhibition of absorption of lumenal cholesterol and bile acids. The inventors have found that this combination provides unexpected and truly synergistic results in terms of reduction of plasma cholesterol. That is, the inventors have discovered that the combination of the cholesterol biosynthesis inhibitor with the soluble fiber provides enhanced cholesterol reduction efficacy relative to the cholesterol biosynthesis inhibitor alone, even wherein the cholesterol biosynthesis inhibitor is utilized at higher levels relative to levels utilized when in combination with the soluble fiber. This is an exciting finding that has potential for revolutionizing associated therapies.

Without intending to be limited by theory, the inventors believe that the soluble fiber sequesters dietary cholesterol, and endogenous cholesterol, bile acids, and other materials which are secreted from the bile, thereby inhibiting absorption of these materials in the plasma. These materials are then passed by the mammalian system, and further endogenous cholesterol must be utilized to generate further bile acid. Thus, again without limitation by theory, it is believed that the cholesterol biosynthesis inhibitor and soluble fiber work synergistically to reduce the levels of cholesterol, in particular LDL cholesterol, in the mammalian system. The compositions, kits, and methods are therefore useful for treating conditions associated with elevated cholesterol, which are defined for simplicity herein as the treatment of atherosclerosis, prevention of atherosclerosis, reduction of plasma cholesterol levels, and combinations thereof.

The components are of the present inventive compositions, as well as those components in the kits and methods (as described further below) are described as follows:

### Cholesterol Biosynthesis Inhibitor

The present compositions comprise a cholesterol biosynthesis inhibitor selected from the group consisting of an HMG CoA reductase inhibitor, an HMG CoA synthase inhibitor, and mixtures thereof.

HMG CoA reductase inhibitors for use herein include, for example lovastatin, pravastatin, fluvastatin, simvastatin, atorvastatin, cerivastatin, and rosuvastatin, all of which shall be interpreted to include the corresponding pharmaceutically-acceptable salts. Lovastatin, pravastatin, simvastatin, atorvastatin, and rosuvastatin are each, individually (or optionally in combination), particularly preferred for use herein. HMG CoA synthase inhibitors for use herein include, for example, (E,E)-11-3′R-(hydroxy-methyl)-4′-oxo-2′R-oxetanyll-3,5,7,R-trimethyl-2,4-undecadienoic acid.

### The Soluble Fiber

Soluble fibers are well-known to those of ordinary skill in the art. Non-limiting examples of soluble fibers include but are not limited to glucomannan (konjac), oat fiber, pectins, psyllium, guar gum, xanthan gum, alginates, gum arabic, fructooligosaccharides (including chicory root and inulin), agar, methylcellulose, and carrageenan. Psyllium (including, for example, psyllium husk or fractionated psyllium) is particularly preferred for use herein. Psyllium husk may be commercially available from The Procter & Gamble Co., Cincinnati, Ohio, U.S.A. Fractionated psyllium has been found to provide a variety of benefits, as described in U.S. Pat. No. 6,287,609. Fraction B or C as described in this patent may be particularly useful as the psyllium herein.

Fructooligosaccharides are also preferred soluble fibers herein. As an example, fructooliogosaccharides are naturally occurring compounds which can be found in a variety of fruits or vegetables including banana, barley, garlic, honey, onion, rye, brown sugar, tomato, asparagus, artichoke, wheat, yacon, or chicory. Fructooligosaccharide may for example be provided as chicory root, as a long chain oligofructose (e.g., inulin), or as short chain oligofructose. Particularly useful herein are fructooligosaccharide comprising at least one of 1-kestose (abbreviated as GF₂), nystose (GF₃), and 1F-beta-fructofuranosylnystose (GE). While fructooligosaccharides can be extracted from plants such as those mentioned herein, they can also be formed artificially by adding one, two, or three fructose units to a sucrose molecule by a B-(2-1)-glycosidic linkage of the fructose unit(s) to the fructose unit of sucrose. As an example, fructooligosaccharides are commercially available under the tradename NUTRAFLORA from Golden Technologies Company, Incorporated (which is a short chain oligofructose comprising 1-kestose, nystose, and 1F-beta-fructofuranosylnystose. As another example, a mixture of short chain fructooligosaccharide and inulin can be PREBIO1 or a mixture of commercially available RAFTILOSE and RAFTILINE.

Preferred pectins include those obtained by hot acidic extraction from citrus peels and may be obtained, for example, from Danisco Co., Braband, Denmark.

Methylcellullose may also be utilized herein, which is the active component of CITRUCEL, commercially available from GlaxoSmithKline, U.S.A.

### Kits of the Present Invention

In yet another embodiment herein, it may be desirable to provide the cholesterol biosynthesis inhibitor and soluble fiber as separate compositions. The invention further relates to kits comprising:

(a) a first composition comprising a cholesterol biosynthesis inhibitor selected from the group consisting of HMG CoA reductase inhibitors, HMG CoA synthase inhibitors, and mixtures thereof; and

(b) a second composition comprising a soluble fiber.

In this embodiment, at least two separate, distinct compositions are provided. The inventors have discovered that such kits are amenable to compliance with treatment regimens which address issues such as disparate dosing frequencies in accordance with optimized embodiments herein, as well as other like factors. In addition, in a particularly preferred embodiment herein, such kits enable essentially continuous, or at least pulsed, availability of the soluble fiber for sequestration of the cholesterol, while the cholesterol biosynthesis inhibitor is available during evening hours (including during or subsequent to the evening meal or prior to the first meal of the subsequent day (e.g., at bedtime)), when cholesterol tends to be synthesized by the mammalian system. As such, the present kits uniquely address the varied mechanisms of the synergistic combination provided herein.

In such kits, various embodiments or preferences of the cholesterol biosynthesis inhibitor and soluble fiber are as described herein above. For simplicity, such embodiments or preferences are not reiterated here.

As an example, wherein the first composition comprises a HMG CoA reductase inhibitor, the HMG CoA reductases inhibitor may be optionally provided as MEVACOR (comprising lovastatin), PRAVACHOL (comprising pravastatin), LESCOL (comprising fluvastatin), ZOCOR (comprising simvastatin), LIPITOR (comprising atorvastatin), or BAYCOR (comprising cerivastatin). Other compositions comprising the cholesterol biosynthesis inhibitor may be formulated in accordance which will be well-known to those of ordinary skill in the art.

As another example, wherein the second composition comprises psyllium, the psyllium may be optionally provided as METAMUCIL, The Procter & Gamble Company, Cincinnati, Ohio, U.S.A. or may otherwise be provided as FIBERALL or PERDIEM. As another example, wherein the second composition comprises methylcellulose, the methylcellulose may be attained as CITRUCEL, GlaxoSmithKline, U.S.A. Other compositions comprising the soluble fiber may be formulated in accordance with methods which will be well-known to those of ordinary skill in the art.

In accordance with this embodiment, the first and second compositions may be present in the kits as separate compositions, e.g., as separate unit dosage forms which are co-packaged, for example, within a containment device, such as for example a carton, bottle, or the like.

In particularly preferred embodiments of the kits herein, the kits comprise a plurality of unit doses of the first composition and/or a plurality of unit doses of the second composition. Optionally, wherein the kits comprise a plurality of unit doses of both the first and second compositions, the plurality of unit doses of the first composition is less than the plurality of unit doses of the second composition. In another embodiment, the number of unit doses of the second composition is from about 2 to about 10 times the number of unit doses of the first composition. In yet another embodiment, the number of unit doses of the second compositions is from about 2 to about 4 times the number of unit doses of the first composition. In yet another embodiment, the number of unit doses of the second compositions is 3 times the number of unit doses of the first composition.

In yet a further embodiment of the present composition, the kits may further comprise information associated with the composition that use of the kit will provide a benefit selected from the group consisting of treatment of atherosclerosis, prevention of atherosclerosis, reduction of plasma cholesterol levels, and combinations thereof. Preferably, such information indicates that one of the benefits described herein will result when the compositions are used in accordance with instructions for use. For example, such directions or instructions for use may include recommended size and frequency of dose, maximum allowable dose, and/or any contraindications.

### Preferred Levels of Soluble Fiber and Cholesterol Biosynthesis Inhibitor

In particularly preferred embodiments herein, the inventors have found that the compositions, unit doses, or kits herein comprise soluble fiber and cholesterol biosynthesis inhibitor at a ratio of at least about 100:1, by weight, alternatively at least about 200:1, by weight, alternatively at least about 250:1 by weight, and further alternatively at least about 300:1 by weight. As has been stated, advantageously the soluble fiber is psyllium and the cholesterol biosynthesis inhibitor is a HMG CoA reductase inhibitor.

Alternatively or additionally, the compositions, unit doses, or kits herein comprise at least about 1 gram of soluble fiber, alternatively at least about 2 grams of soluble fiber, alternatively at least about 3 grams of soluble fiber, alternatively about 5 grams of soluble fiber, alternatively from about 1 gram to about 20 grams of soluble fiber, alternatively from about 2 grams to about 17 grams of soluble fiber, alternatively from about 3 grams to about 15 grams of soluble fiber, and alternatively from about 4 grams to about 7 grams of soluble fiber.

Alternatively or additionally, the compositions, unit doses, or kits herein comprise at least about 1 mg of cholesterol biosynthesis inhibitor, alternatively at least about 2 mg of cholesterol biosynthesis inhibitor, alternatively at least about 5 mg of cholesterol biosynthesis inhibitor, alternatively from about 1 mg to about 100 mg of cholesterol biosynthesis inhibitor, alternatively from about 2 mg to about 80 mg of cholesterol biosynthesis inhibitor, and alternatively from about 5 mg to about 80 mg of cholesterol biosynthesis inhibitor.

### Optional Components and Dose Forms of the Present Compositions and Unit Doses

The compositions described herein may be administered concurrently with other materials, or ingested separately as part of a dosing regimen during a treatment period.

A non-limiting description of suitable excipients and/or other adjuvants is provided in the “Inactive Ingredient Guide” published by the U.S. Food and Drug Administration (see, for example, hitp://www.fda.gov/cder/drug/iig).cd

The compositions described herein may be administered in any convenient form including, for example, a capsule, tablet (including swallowable or chewable forms), suspension, suppository, powders (including such powders which are suitable for admixture with a liquid such as, for example, water or juice), or the like. Wherein the cholesterol biosynthesis inhibitor and soluble fiber are administered as separate compositions, the unit dose form of each may be independent of the other. For example, the cholesterol biosynthesis inhibitor may be in a unit dose form that is a capsule or caplet, while the soluble fiber may be in a unit dose form which is a capsule or powder.

### Methods of the Present Invention

The present methods are useful for a variety of purposes that are related to the treatment (including treatment, prevention and/or inhibition) of conditions associated with elevated cholesterol levels. Such conditions include, but are not limited to, one or more of the following: cardiovascular conditions including, but not limited to, atherosclerosis (including coronary heart disease), restenosis, thrombosis, hypercholesterolemia, hypertension, risk of heart attack, diabetes, vascular dysfunction, and poor circulation, and other conditions such as shock. Preferred methods herein include treatment of one or more of atherosclerosis, hypercholesterolemia, hypertension, risk of heart attack, diabetes, and poor circulation. Ancillary treatments or benefits by virtue of utilization of the soluble fiber herein will of course include treatment of gastrointestinal conditions typically treated through use of a soluble fiber.

Such methods comprise systemically (typically, orally) administering to a mammal (preferably, a human) successive therapeutically effective doses of the compositions described herein. In particular, the present methods comprising administering to a mammal in need of treatment a composition comprising a cholesterol biosynthesis inhibitor and a soluble fiber, or separate compositions comprising a first composition comprising a cholesterol biosynthesis inhibitor and a second composition comprising a soluble fiber.

The methods of the present invention comprise administration (typically, oral) of the cholesterol biosynthesis inhibitor and the soluble fiber, either as separate unit doses (e.g., the first composition and the second composition, as described herein above with respect to the kits) or concurrently as a single composition (as also described herein), to a mammal (most preferably a human). Frequency of administration is not limited, however, the compositions described herein are typically administered on an infrequent or as-needed basis or may be administered in a more routine manner daily, or on a more or less frequent basis.

For example, the compositions described herein may be administered once daily or with meals. It is typical to dose the compositions, particularly those comprising a cholesterol biosynthesis inhibitor, in the evening hours (including during or subsequent to the evening meal or prior to the first meal of the subsequent day (e.g., at bedtime)), at times when cholesterol biosynthesis peaks. Alternatively or additionally, the compositions may be dosed early in the morning, particularly those comprising a soluble fiber, as bile may be most concentrated with endogenous cholesterol in the morning. Wherein the components are administered separately, the components may be dosed at various times or frequencies.

For example, optionally, it may be particularly advantageous to dose a composition comprising the soluble fiber two or three times daily, with meals, while the composition comprising the cholesterol biosynthesis inhibitor may optionally be administered only once daily. In general, compositions comprising the cholesterol biosynthesis inhibitor are administered at least once monthly, more typically at least once weekly, more typically at least once daily. Also in general, compositions comprising the soluble fiber are administered at least once monthly, more typically at least once weekly, more typically at least once daily, even more typically at least twice daily, or even more typically at least three times daily. In a preferred embodiment, wherein the cholesterol biosynthesis inhibitor and soluble fiber are administered as separate compositions, the compositions comprising the cholesterol biosynthesis inhibitor are administered once daily. Alternatively or additionally, wherein the cholesterol biosynthesis inhibitor and soluble fiber are administered as separate compositions, the compositions comprising the soluble fiber are administered at least once daily, or at least twice daily, or at least three times daily. In one embodiment, at least one unit dose of the composition comprising the soluble fiber is administered concurrently with the composition comprising the cholesterol biosynthesis inhibitor.

As used herein, the term “administer,” “administration,” or the like with regard to a particular composition means to provide the composition to the mammal (including oneself) and/or to direct, instruct, or advise the use of the composition for any purpose (preferably, for a purpose described herein). Wherein the administration of one or more of the present compositions is directed, instructed or advised, such direction may be that which instructs and/or informs the user that use of the composition may and/or will provide one or more of the benefits described herein. Non-limiting examples of such instruction or information are set forth herein as part of the description of the present kits.

Administration which is directed may comprise, for example, oral direction (e.g., through oral instruction from, for example, a physician, health professional, sales professional or organization, and/or radio or television media (i.e., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or packaging associated with the composition (e.g., a label present on a package containing the composition). As used herein, “written” includes through words, pictures, symbols, and/or other visible descriptors. Such direction need not utilize the actual words used herein, but rather use of words, pictures, symbols, and the like conveying the same or similar meaning are contemplated within the scope of this invention.

As used herein, the term “safe and effective amount” of a component, composition, or like material is an amount that is effective for the treatment of conditions associated with elevated cholesterol levels in a mammal (preferably a human), without undue adverse side effects (such as toxicity, irritation, or allergic response), commensurate with a reasonable benefit/risk ratio when used in the manner of this invention. The specific “safe and effective amount” will, obviously, vary with such factors as the particular condition being treated, the physical condition of the treated mammal, the size and weight of the treated animal, the duration of treatment, the nature of concurrent therapy (if any), the specific dosage form to be used, other components present in a given dosed composition, and the dosage regimen desired for the component or composition.

### In Vivo Assays of Compositions Described Herein

The in vivo activity of the presently described compositions, as well as treatment utilization of kits and treatment methods, may be optionally determined by either of the following procedures.

Male dogs (beagles, ranging from about 9 to about 14 kilograms, 1 to 4 years old) are fed a standard dog feed supplemented with 5.5% lard and 1% cholesterol. Baseline blood samples are drawn from fasted dogs prior to initiating the study to obtain reference values for plasma cholesterol. Dogs are then randomized to groups of five animals with similar plasma cholesterol levels. The animals are dosed in accordance with a treatment method described herein immediately prior to diet presentation for seven days. Blood samples are obtained 24 hours after the last dose for plasma cholesterol determinations. Plasma cholesterol levels are determined by a modification of the cholesterol oxidase method using a commercially available kit.

In an optional alternative procedure, hamsters are separated into groups of six and given a controlled cholesterol diet containing 0.5% cholesterol for seven days. Diet consumption is monitored to determine dietary cholesterol exposure. The animals are dosed in accordance with a treatment method described herein once daily beginning with the initiation of diet. Dosing is by oral gavage. All animals moribund or in poor physical condition are euthanized. After seven days, the animals are anesthetized by intramuscular (IM) injection of ketamine and sacrificed by decapitation. Blood is collected into vacutainer tubes containing EDTA for plasma lipid analysis and the liver is excised for tissue lipid analysis. Lipid analysis is conducted as per published procedures (e.g., Schnitzer-Polokoff et al., Comp. Biochem. Physiol., 99A, 4 (1991), pp. 665-670 and data is recorded as percent reduction of lipid versus control.

### Non-Limiting Examples of the Present Invention

The following are non-limiting examples of the presently described compositions, kits, and methods. The described compositions are prepared utilizing conventional processes or, in the case of separate, distinct compositions may be otherwise commercially available. The examples are provided to illustrate the invention and are not intended to limit the scope thereof in any manner.

### EXAMPLE 1

A kit is provided comprising a 14-day supply of 14 unit doses of a first composition comprising simvastatin (10 mg per unit dose, each as a tablet further comprising excipients such as one or more of cellulose, lactose, magnesium stearate, iron oxide, talc, titanium dioxide, and starch) and a second composition, in bulk, comprising 42 unit doses of psyllium (as a bulk powder further comprising excipients such as one or more of maltodextrin, citric acid, flavors, colors, and aspartame). At the time of ingestion of each unit dose of the second composition, the human male meters 5 grams of bulk powder per each unit dose and admixes such 5 grams of bulk powder with 8 ounces of a ready-to-drink fruit juice or water. A human male suffering from elevated plasma cholesterol levels, and advised of being at risk for heart attack, orally ingests one unit dose of the first composition and three unit doses of the second composition, daily. After utilization of four kits, the human male exhibits an approximate 30% decrease in LDL-cholesterol as measured and reported by a physician.

### EXAMPLE 2

Pravastatin sodium (10 mg) is admixed with methylcellulose (2 grams) and the resulting mixture is filled along with standard excipients into a soft gelatin capsule.

### EXAMPLE 3

A kit is provided comprising a 28-day supply of 28 unit doses of a first composition comprising atorvastatin (20 mg per unit dose, each as a tablet further comprising excipients such as one or more of cellulose, lactose, magnesium stearate, iron oxide, talc, titanium dioxide, and starch) and 84 unit doses of a second composition comprising psyllium (1 gram per unit dose, each as a gelatin capsule). A human female suffering from elevated plasma cholesterol levels, and having a history of heart attack) orally ingests one unit dose of the first composition and three unit doses of the second composition, daily. After utilization of two kits, the human female exhibits an approximate 25% decrease in total cholesterol as measured and reported by a physician.

### EXAMPLE 4

A comparative study is executed to determine the effects of methods of treating elevated cholesterol levels comprising administration of each of: Test Sample 1, simvastatin (20 mg) in conjunction with fruit juice; Test Sample 2, simvastatin (10 mg) in conjunction with fruit juice; and Test Sample 3, simvastatin (10 mg) in conjunction with psyllium (15 grams) and fruit juice.

The study is a double-blinded, randomized comparison. Sixty humans, aging from about 30 to about 80 years, are utilized for the study, all of which are determined as having risk factors for atherosclerosis. The humans are randomized to three treatment groups (Treatment Group 1, Treatment Group 2, and Treatment Group 3). Each human stops any lipid-lowering treatment, and baseline lipid levels are obtained.

Treatment Group 1 receives Test Sample 1; Treatment Group 2 receives Test Sample 2; and Treatment Group 3 receives Test Sample 3; all over an eight week period. Test Sample 1 is administered as a concurrent administration once daily. Test Sample 2 is administered as a concurrent administration once daily. For Test Sample 3, 15 grams of psyllium is divided over three daily doses, each with fruit juice, wherein the last administered dose per day is concurrently administered with the simvastatin.

At eight weeks, levels of LDL-cholesterol, total cholesterol, and triglycerides are measured. Triglycerides are found not to be influenced based upon treatment group. However, on average, LDL-cholesterol and total cholesterol is decreased by approximately 29% in Treatment Group 1, LDL-cholesterol and total cholesterol is decreased by approximately 33% in Treatment Group 2, and LDL-cholesterol and total cholesterol is decreased by approximately 38% in Treatment Group 3.

What is claimed is:

1. A method of lowering the total cholesterol level in a human comprising administering to a human
a. at least one unit dose of cholesterol biosynthesis inhibitor selected from the group consisting of HMG CoA reductase inhibitors, HMG CoA synthase inhibitors, and mixtures thereof;
b. a first unit dose of soluble fiber;
c. a second unit dose of soluble fiber; and
d. a third unit dose of soluble fiber;
wherein the total cholesterol level is lowered.

2. The method according to claim 1 wherein the cholesterol biosynthesis inhibitor is an HMG CoA reductase inhibitor selected from the group consisting of lovastatin, pravastatin, simvastatin, atorvastatin, and mixtures thereof.

3. The method according to claim 1 wherein the unit dose of cholesterol biosynthesis inhibitor comprises from 5 milligrams to 80 milligrams of cholesterol biosynthesis inhibitor.

4. The method according to claim 3 wherein the unit dose of cholesterol biosynthesis inhibitor comprises from 10 milligrams to 20 milligrams of cholesterol biosynthesis inhibitor.

5. The method according to claim 1 wherein the first unit dose of soluble fiber, the second unit dose of soluble fiber, and the third unit dose are selected from the group consisting of fructooligosaccharides, psyllium, oat fiber, and combinations thereof.

6. The method according to claim 1 wherein the first unit dose of soluble fiber, the second unit dose of soluble fiber, and the third unit dose of soluble fiber comprise psyllium.

7. The method according to claim 1 wherein the first unit dose of soluble fiber, the second unit dose of soluble fiber, and the third unit dose of soluble fiber comprise at least one gram of soluble fiber.

8. The method according to claim 1 wherein the first unit dose of soluble fiber, the second unit dose of soluble fiber, and the third unit dose of soluble fiber comprise at least two grams of soluble fiber.

9. The method according to claim 1 wherein the first unit dose of soluble fiber, the second unit dose of soluble fiber, and the third unit dose of soluble fiber comprise at least three grams of soluble fiber.

10. The method according to claim 1 wherein the unit dose of cholesterol biosynthesis inhibitor, the first unit dose of soluble fiber, the second unit dose of soluble fiber, and the third unit dose of soluble fiber are administered concurrently.

11. The method according to claim 1 wherein the first unit dose of soluble fiber, the second unit dose of soluble fiber, and the third unit dose of soluble fiber are administered two or three times daily.

12. The method according to claim 11 wherein the first unit dose of soluble fiber, the second unit dose of soluble fiber, and the third unit dose of soluble fiber are administered with meals and the unit dose of cholesterol biosynthesis inhibitor is administered once daily.

13. The method according to claim 12 wherein the unit dose of cholesterol biosynthesis inhibitor and the third unit dose of soluble fiber are administered concurrently in the evening hours.

14. The method according to claim 1 wherein the cholesterol is reduced by at least 25%.

15. The method according to claim 1 wherein the cholesterol is reduced at least about 30%.

16. The method according to claim 1 wherein the administration occurs for at least 14 days.

17. The method according to claim 1 wherein the administration occurs for at least 28 days.

18. The method according to claim 1 wherein the unit dose of cholesterol biosynthesis inhibitor and the third unit dose of soluble fiber are a single composition.

19. A method of lowering the total cholesterol level in a human comprising administering to a human
a. from 5 milligrams to 80 milligrams of a HMG CoA reductase inhibitor selected from the group consisting of lovastatin, pravastatin, simvastatin, atorvastatin, and mixtures thereof, wherein the HMG CoA reductase inhibitor is administered one time daily in the evening;
b. a first unit dose of psyllium comprising from 1 gram to 3 grams of psyllium administered at a first time;
c. a second unit dose of psyllium comprising from 1 gram to 3 grams of psyllium administered at a second time; and
d. a third unit dose of psyllium comprising from 1 gram to 3 grams of psyllium wherein the third unit dose of psyllium is administered concurrently with the HMG CoA reductase inhibitor during the evening hours; and
wherein the total cholesterol level is lowered by at least 25%.

20. The method of claim 19 comprising from about 10 milligrams to about 20 milligrams of the HMG CoA reductase inhibitor, 3 grams of a first unit dose of psyllium, 3 grams of a second unit dose of psyllium, and 3 grams of a third unit dose of psyllium.
